(19) United States
(12) Patent Application Publication
(10) Pub. No.: US 2026/0363611 A1
(43) Pub. Date: Nov. 19, 2026

(54) REAL-TIME SLEEP STAGING PREDICTIONS USING LEARNED SLEEP STRUCTURES

(71) Applicant: Samsung Electronics Co., Ltd., Suwon-si (KR)

(72) Inventors: Viswam Nathan, Clovis, CA (US); Keum San Chun, Sunnyvale, CA (US); Varun Kumar Viswanath, Fremont, CA (US); Jilong Kuang, San Jose, CA (US)

(21) Appl. No.: 19/665,860

(22) Filed: May 1, 2026

Related U.S. Application Data
(60) Provisional application No. 63/805,100, filed on May 13, 2025.

(51) Int. Cl. A61B 5/00; A61B 5/024; A61B 5/08; A61B 5/11; G06N 20/00

(52) U.S. Cl. CPC A61B 5/4812; A61B 5/02405; A61B 5/0816; A61B 5/11; G06N 20/00

(57) ABSTRACT

A method includes obtaining physiological information associated with a subject, where the physiological information is divided into epochs associated with different periods of time. The method also includes generating features of the physiological information, providing the features to a trained machine learning model, and generating real-time sleep stage predictions of the subject during the epochs using the machine learning model. The machine learning model generates the real-time sleep stage predictions for at least one current epoch using the features associated with the at least one current epoch and the features associated with one or more prior epochs. The real-time sleep stage predictions for the at least one current epoch include a predicted sleep stage of the subject during the at least one current epoch and at least one predicted future sleep stage of the subject during one or more subsequent epochs.

### CROSS-REFERENCE TO RELATED APPLICATION AND PRIORITY CLAIM

This application claims priority under 35 U.S.C. § 119(e) to U.S. Provisional Patent Application No. 63/805,100 filed on May 13, 2025. This provisional patent application is hereby incorporated by reference in its entirety.

### TECHNICAL FIELD

This disclosure relates generally to signal processing systems and processes. More specifically, this disclosure relates to real-time sleep staging predictions using learned sleep structures.

### BACKGROUND

Wearable devices, such as smart watches and smart rings, are becoming more and more popular and are often worn by users during sleep. Other devices, such as visible or infrared cameras, can be used to monitor people during sleep. As a result, these devices provide an opportunity to collect sleep-related information about people, and that information can be used to derive health predictions, provide advice to the people, or perform other functions. For example, algorithms could be used to analyze people’s sleep-related information and identify potential sleep interventions, which could be applied in order to improve the people’s sleep quality and morning cognitive abilities.

### SUMMARY

This disclosure relates to real-time sleep staging predictions using learned sleep structures.

In a first embodiment, a method includes obtaining, using at least one processing device, physiological information associated with a subject, where the physiological information is divided into epochs associated with different periods of time. The method also includes generating, using the at least one processing device, features of the physiological information. The method further includes providing, using the at least one processing device, the features to a trained machine learning model. In addition, the method includes generating, using the at least one processing device, real-time sleep stage predictions of the subject during the epochs using the trained machine learning model. The trained machine learning model generates the real-time sleep stage predictions for at least one current epoch using the features associated with the at least one current epoch and the features associated with one or more prior epochs. The real-time sleep stage predictions for the at least one current epoch include (i) a predicted sleep stage of the subject during the at least one current epoch and (ii) at least one predicted future sleep stage of the subject during one or more subsequent epochs following the at least one current epoch.

In a second embodiment, an apparatus includes at least one processing device configured to obtain physiological information associated with a subject, where the physiological information is divided into epochs associated with different periods of time. The at least one processing device is also configured to generate features of the physiological information, provide the features to a trained machine learning model, and generate real-time sleep stage predictions of the subject during the epochs using the trained machine learning model. The trained machine learning model is configured to generate the real-time sleep stage predictions for at least one current epoch using the features associated with the at least one current epoch and the features associated with one or more prior epochs. The real-time sleep stage predictions for the at least one current epoch include (i) a predicted sleep stage of the subject during the at least one current epoch and (ii) at least one predicted future sleep stage of the subject during one or more subsequent epochs following the at least one current epoch.

Any one or any combination of the following features may be used with the first or second embodiment. The epochs may be defined by a repeating time interval, the features may be generated for different collections of time intervals using the physiological information collected during the different collections of time intervals, and the trained machine learning model may (for at least some of the time intervals) process the features associated with the at least one current epoch and the one or more prior epochs and generate real-time predictions of sleep stages of the subject during the at least one current epoch and the one or more subsequent epochs. The trained machine learning model may be trained to recognize transitions between different sleep stages of people and an overall structure of sleep by the people. Polysomnography data may be used as ground truth data or to generate the ground truth data during training of the trained machine learning model so that the trained machine learning model can learn to label the epochs with the real-time sleep stage predictions. The physiological information may include heart rate information of the subject, heart rate variability information of the subject, breathing rate information of the subject, movement information of the subject, or a combination thereof. The physiological information may be preprocessed to generate preprocessed physiological information, the features may be generated using the preprocessed physiological information, and the physiological information may be preprocessed by removing noise from the physiological information and/or normalizing the physiological information. Operation of an electronic device or at least one wearable device associated with the subject may be modified or controlled based on at least one of the predicted sleep stage of the subject during the current epoch or the at least one predicted future sleep stage of the subject during the one or more subsequent epochs.

A non-transitory machine readable medium may contain instructions that when executed cause at least one processing device of an electronic device to perform the method of the first embodiment, optionally along with any feature or combination of features discussed above.

In a third embodiment, a method includes obtaining, using at least one processing device of an electronic device, training data that includes (i) features of physiological information associated with people (where the physiological information is divided into epochs associated with different periods of time) and (ii) ground truth data identifying sleep stages of the people. The method also includes training, using the at least one processing device, a machine learning model based on the training data. The machine learning model is trained to repeatedly predict, based on features of physiological information associated with a specified subject during at least one current epoch and one or more prior epochs, (i) a current sleep stage of the specified subject during the at least one current epoch and (ii) one or more predicted future sleep stages of the specified subject during one or more subsequent epochs following the at least one current epoch.

Any one or any combination of the following features may be used with the third embodiment. The machine learning model may be trained to recognize transitions between different sleep stages of the people and an overall structure of sleep by the people. The machine learning model may be trained by providing the features of the physiological information associated with the people to the machine learning model, generating predictions of sleep stages of the people using the machine learning model based on the features of the physiological information associated with the people, comparing the predictions to the ground truth data in order to estimate a loss, and adjusting the machine learning model until the loss is below a threshold. The machine learning model may be trained, for at least some of the epochs associated with the specified subject, to process the features associated with the at least one current epoch and the one or more prior epochs and generate real-time predictions of sleep stages of the specified subject during the at least one current epoch and the one or more subsequent epochs. Polysomnography data may be used as the ground truth data or to generate the ground truth data during the training of the machine learning model. The physiological information may include heart rate information of the people, heart rate variability information of the people, breathing rate information of the people, movement information of the people, or a combination thereof.

An electronic device may include at least one processing device configured to perform the method of the third embodiment, optionally along with any feature or combination of features discussed above. A non-transitory machine readable medium may contain instructions that when executed cause at least one processing device of an electronic device to perform the method of the third embodiment, optionally along with any feature or combination of features discussed above.

Other technical features may be readily apparent to one skilled in the art from the following figures, descriptions, and claims.

Before undertaking the DETAILED DESCRIPTION below, it may be advantageous to set forth definitions of certain words and phrases used throughout this patent document. The terms “transmit,” “receive,” and “communicate,” as well as derivatives thereof, encompass both direct and indirect communication. The terms “include” and “comprise,” as well as derivatives thereof, mean inclusion without limitation. The term “or” is inclusive, meaning and/or. The phrase “associated with,” as well as derivatives thereof, means to include, be included within, interconnect with, contain, be contained within, connect to or with, couple to or with, be communicable with, cooperate with, interleave, juxtapose, be proximate to, be bound to or with, have, have a property of, have a relationship to or with, or the like.

Moreover, various functions described below can be implemented or supported by one or more computer programs, each of which is formed from computer readable program code and embodied in a computer readable medium. The terms “application” and “program” refer to one or more computer programs, software components, sets of instructions, procedures, functions, objects, classes, instances, related data, or a portion thereof adapted for implementation in a suitable computer readable program code. The phrase “computer readable program code” includes any type of computer code, including source code, object code, and executable code. The phrase “computer readable medium” includes any type of medium capable of being accessed by a computer, such as read only memory (ROM), random access memory (RAM), a hard disk drive, a compact disc (CD), a digital video disc (DVD), or any other type of memory. A “non-transitory” computer readable medium excludes wired, wireless, optical, or other communication links that transport transitory electrical or other signals. A non-transitory computer readable medium includes media where data can be permanently stored and media where data can be stored and later overwritten, such as a rewritable optical disc or an erasable memory device.

As used here, terms and phrases such as “have,” “may have,” “include,” or “may include” a feature (like a number, function, operation, or component such as a part) indicate the existence of the feature and do not exclude the existence of other features. Also, as used here, the phrases “A or B,” “at least one of A and/or B,” or “one or more of A and/or B” may include all possible combinations of A and B. For example, “A or B,” “at least one of A and B,” and “at least one of A or B” may indicate all of (1) including at least one A, (2) including at least one B, or (3) including at least one A and at least one B. Further, as used here, the terms “first” and “second” may modify various components regardless of importance and do not limit the components. These terms are only used to distinguish one component from another. For example, a first user device and a second user device may indicate different user devices from each other, regardless of the order or importance of the devices. A first component may be denoted a second component and vice versa without departing from the scope of this disclosure.

It will be understood that, when an element (such as a first element) is referred to as being (operatively or communicatively) “coupled with/to” or “connected with/to” another element (such as a second element), it can be coupled or connected with/to the other element directly or via a third element. In contrast, it will be understood that, when an element (such as a first element) is referred to as being “directly coupled with/to” or “directly connected with/to” another element (such as a second element), no other element (such as a third element) intervenes between the element and the other element.

As used here, the phrase “configured (or set) to” may be interchangeably used with the phrases “suitable for,” “having the capacity to,” “designed to,” “adapted to,” “made to,” or “capable of” depending on the circumstances. The phrase “configured (or set) to” does not essentially mean “specifically designed in hardware to.” Rather, the phrase “configured to” may mean that a device can perform an operation together with another device or parts. For example, the phrase “processor configured (or set) to perform A, B, and C” may mean a generic-purpose processor (such as a CPU or application processor) that may perform the operations by executing one or more software programs stored in a memory device or a dedicated processor (such as an embedded processor) for performing the operations.

The terms and phrases as used here are provided merely to describe some embodiments of this disclosure but not to limit the scope of other embodiments of this disclosure. It is to be understood that the singular forms “a,” “an,” and “the” include plural references unless the context clearly dictates otherwise. All terms and phrases, including technical and scientific terms and phrases, used here have the same meanings as commonly understood by one of ordinary skill in the art to which the embodiments of this disclosure belong. It will be further understood that terms and phrases, such as those defined in commonly-used dictionaries, should be interpreted as having a meaning that is consistent with their meaning in the context of the relevant art and will not be interpreted in an idealized or overly formal sense unless expressly so defined here. In some cases, the terms and phrases defined here may be interpreted to exclude embodiments of this disclosure.

Examples of an “electronic device” according to embodiments of this disclosure may include at least one of a smartphone, a tablet personal computer (PC), a mobile phone, a video phone, an e-book reader, a desktop PC, a laptop computer, a netbook computer, a workstation, a personal digital assistant (PDA), a portable multimedia player (PMP), an MP3 player, a mobile medical device, a camera, or a wearable device (such as smart glasses, a head-mounted device (HMD), electronic clothes, an electronic bracelet, an electronic necklace, an electronic accessory, an electronic tattoo, a smart mirror, or a smart watch). Other examples of an electronic device include a smart home appliance. Examples of the smart home appliance may include at least one of a television, a digital video disc (DVD) player, an audio player, a refrigerator, an air conditioner, a cleaner, an oven, a microwave oven, a washer, a dryer, an air cleaner, a set-top box, a home automation control panel, a security control panel, a TV box (such as SAMSUNG HOMESYNC, APPLETV, or GOOGLE TV), a smart speaker or speaker with an integrated digital assistant (such as SAMSUNG GALAXY HOME, APPLE HOMEPOD, or AMAZON ECHO), a gaming console (such as an XBOX, PLAYSTATION, or NINTENDO), an electronic dictionary, an electronic key, a camcorder, or an electronic picture frame. Still other examples of an electronic device include at least one of various medical devices (such as diverse portable medical measuring devices (like a blood sugar measuring device, a heartbeat measuring device, or a body temperature measuring device), a magnetic resonance angiography (MRA) device, a magnetic resonance imaging (MRI) device, a computed tomography (CT) device, an imaging device, or an ultrasonic device), a navigation device, a global positioning system (GPS) receiver, an event data recorder (EDR), a flight data recorder (FDR), an automotive infotainment device, a sailing electronic device (such as a sailing navigation device or a gyro compass), avionics, security devices, vehicular head units, industrial or home robots, automatic teller machines (ATMs), point of sales (POS) devices, or Internet of Things (IoT) devices (such as a bulb, various sensors, electric or gas meter, sprinkler, fire alarm, thermostat, street light, toaster, fitness equipment, hot water tank, heater, or boiler). Other examples of an electronic device include at least one part of a piece of furniture or building/structure, an electronic board, an electronic signature receiving device, a projector, or various measurement devices (such as devices for measuring water, electricity, gas, or electromagnetic waves). Note that, according to various embodiments of this disclosure, an electronic device may be one or a combination of the above-listed devices. According to some embodiments of this disclosure, the electronic device may be a flexible electronic device. The electronic device disclosed here is not limited to the above-listed devices and may include any other electronic devices now known or later developed.

In the following description, electronic devices are described with reference to the accompanying drawings, according to various embodiments of this disclosure.

Definitions for other certain words and phrases may be provided throughout this patent document. Those of ordinary skill in the art should understand that in many if not most instances, such definitions apply to prior as well as future uses of such defined words and phrases.

None of the description in this application should be read as implying that any particular element, step, or function is an essential element that must be included in the claim scope. The scope of patented subject matter is defined only by the claims. Moreover, none of the claims is intended to invoke 35 U.S.C. § 112(f) unless the exact words “means for” are followed by a participle. Use of any other term, including without limitation “mechanism,” “module,” “device,” “unit,” “component,” “element,” “member,” “apparatus,” “machine,” “system,” “processor,” or “controller,” within a claim is understood by the Applicant to refer to structures known to those skilled in the relevant art and is not intended to invoke 35 U.S.C. § 112(f).

### BRIEF DESCRIPTION OF THE DRAWINGS

For a more complete understanding of this disclosure and its advantages, reference is now made to the following description taken in conjunction with the accompanying drawings, in which:

FIG. 1 illustrates an example network configuration including an electronic device in accordance with this disclosure;

FIG. 2 illustrates an example process for generating real-time sleep staging predictions using learned sleep structures in accordance with this disclosure;

FIGS. 3 - 5 illustrate example architectures for generating real-time sleep staging predictions using learned sleep structures in accordance with this disclosure;

FIG. 6 illustrates an example method for generating real-time sleep staging predictions using learned sleep structures in accordance with this disclosure; and

FIG. 7 illustrates an example method for training a machine learning model to generate real-time sleep staging predictions using learned sleep structures in accordance with this disclosure.

### DETAILED DESCRIPTION

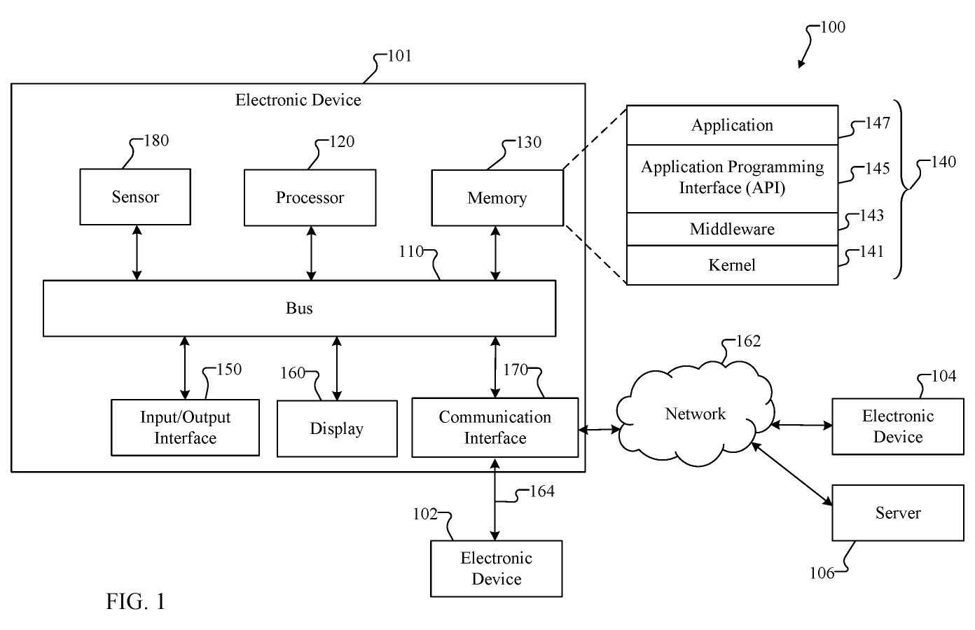

FIGS. 1 - 7, discussed below, and the various embodiments of this disclosure are described with reference to the accompanying drawings. However, it should be appreciated that this disclosure is not limited to these embodiments, and all changes and/or equivalents or replacements thereto also belong to the scope of this disclosure. The same or similar reference denotations may be used to refer to the same or similar elements throughout the specification and the drawings.

As noted above, wearable devices, such as smart watches and smart rings, are becoming more and more popular and are often worn by users during sleep. Other devices, such as visible or infrared cameras, can be used to monitor people during sleep. As a result, these devices provide an opportunity to collect sleep-related information about people, and that information can be used to derive health predictions, provide advice to the people, or perform other functions. For example, algorithms could be used to analyze people’s sleep-related information and identify potential sleep interventions, which could be applied in order to improve the people’s sleep quality and morning cognitive abilities.

Algorithms used to predict the stages of people’s sleep are often based on physiological information, such as heart rate, breathing rate, and body movements. By using this physiological information, the algorithms attempt to predict people’s sleep stages throughout the night. However, the most-precise sleep staging algorithms currently run after the fact, meaning they use an entire night of data to predict all of the sleep stages of a person during that night. While this allows these algorithms to estimate how a person enters progressively-deeper stages of sleep over time, this precludes the generation of real-time sleep stage predictions.

Current real-time sleep staging algorithms generally operate to predict a person’s current sleep stage based on differences in motion, heart rate, heart rate variability, and breathing rate between different sleep stages. By analyzing information collected during multiple blocks of time, these algorithms attempt to label each block with a specific sleep stage. However, these approaches are based on the assumption that each block of time is independent. In reality, it has been discovered that sleep follows a high-level structure composed of multiple sleep cycles where, within each cycle, people enter progressively-deeper stages of sleep. In other words, a person’s sleep typically has a high-level structure in which a given sleep stage is not entirely independent of the preceding sleep stage(s). As sleep cycles occur over the course of multiple hours, algorithms that independently process information in blocks of time are unlikely to learn or use the high-level structure of a person’s sleep. Moreover, these approaches estimate a person’s sleep stage for each block of time without attempting to predict the person’s future sleep stage(s) during one or more subsequent blocks of time. Overall, these issues can result in significantly-reduced accuracy of sleep stage predictions.

This disclosure provides various techniques supporting real-time sleep staging predictions using learned sleep structures. As described in more detail below, physiological information associated with a subject (such as physiological information collected by at least one wearable device worn by the subject) can be obtained. The physiological information can be divided into epochs associated with different periods of time. Features of the physiological information can be generated and provided to a trained machine learning model, and real-time sleep stage predictions of the subject during the epochs can be generated using the trained machine learning model. The trained machine learning model can generate the real-time sleep stage predictions for at least one current epoch using the features associated with the at least one current epoch and the features associated with one or more prior epochs. The real-time sleep stage predictions for the at least one current epoch can include (i) a predicted sleep stage of the subject during the at least one current epoch and (ii) at least one predicted future sleep stage of the subject during one or more subsequent epochs following the at least one current epoch.

Also, as described in more detail below, training data can be obtained, and the training data can include (i) features of physiological information associated with people (where the physiological information is divided into epochs associated with different periods of time) and (ii) ground truth data identifying sleep stages of the people. A machine learning model can be trained based on the training data. The machine learning model can be trained to repeatedly predict, based on features of physiological information associated with a specified subject during at least one current epoch and one or more prior epochs, (i) a current sleep stage of the specified subject during the at least one current epoch and (ii) one or more predicted future sleep stages of the specified subject during one or more subsequent epochs following the at least one current epoch.

In this way, the described techniques support more accurate predictions of people’s sleep stages and therefore improved recommendations, analyses, or other actions based on those predictions. For example, the described techniques can leverage long-context inputs of multiple prior epochs of sleep data to make predictions of current and future sleep stages, rather than processing the sleep data of each epoch independently. As a result, the described techniques can support a data-driven approach for encoding the temporal structure of a person’s sleep into a real-time sleep staging algorithm. Moreover, the described techniques allow a machine learning model to learn the high-level structure of a person’s sleep so that knowledge of how one sleep stage is dependent on a preceding stage can be exploited. In addition, the machine learning model can be trained using a model training objective that involves learning to predict future sleep stages (rather than just a current sleep stage), which can force the machine learning model to learn the high-level structure governing transitions between multiple sleep stages. Overall, these techniques can help to improve the performance of sleep stage classification.

FIG. 1 illustrates an example network configuration 100 including an electronic device in accordance with this disclosure. The embodiment of the network configuration 100 shown in FIG. 1 is for illustration only. Other embodiments of the network configuration 100 could be used without departing from the scope of this disclosure.

According to embodiments of this disclosure, an electronic device 101 is included in the network configuration 100. The electronic device 101 can include at least one of a bus 110, a processor 120, a memory 130, an input/output (I/O) interface 150, a display 160, a communication interface 170, and a sensor 180. In some embodiments, the electronic device 101 may exclude at least one of these components or may add at least one other component. The bus 110 includes a circuit for connecting the components 120-180 with one another and for transferring communications (such as control messages and/or data) between the components.

The processor 120 includes one or more processing devices, such as one or more microprocessors, microcontrollers, digital signal processors (DSPs), application specific integrated circuits (ASICs), or field programmable gate arrays (FPGAs). In some embodiments, the processor 120 includes one or more of a central processing unit (CPU), an application processor (AP), a communication processor (CP), a graphics processor unit (GPU), or a neural processing unit (NPU). The processor 120 is able to perform control on at least one of the other components of the electronic device 101 and/or perform an operation or data processing relating to communication or other functions. As described below, the processor 120 may perform one or more functions related to real-time sleep staging predictions using learned sleep structures, such as by training machine learning models and/or using trained machine learning models for real-time sleep staging predictions.

The memory 130 can include a volatile and/or non-volatile memory. For example, the memory 130 can store commands or data related to at least one other component of the electronic device 101. According to embodiments of this disclosure, the memory 130 can store software and/or a program 140. The program 140 includes, for example, a kernel 141, middleware 143, an application programming interface (API) 145, and/or an application program (or “application”) 147. At least a portion of the kernel 141, middleware 143, or API 145 may be denoted an operating system (OS).

The kernel 141 can control or manage system resources (such as the bus 110, processor 120, or memory 130) used to perform operations or functions implemented in other programs (such as the middleware 143, API 145, or application 147). The kernel 141 provides an interface that allows the middleware 143, the API 145, or the application 147 to access the individual components of the electronic device 101 to control or manage the system resources. The application 147 may include one or more applications that, among other things, perform functions related to real-time sleep staging predictions using learned sleep structures. These functions can be performed by a single application or by multiple applications that each carries out one or more of these functions. The middleware 143 can function as a relay to allow the API 145 or the application 147 to communicate data with the kernel 141, for instance. A plurality of applications 147 can be provided. The middleware 143 is able to control work requests received from the applications 147, such as by allocating the priority of using the system resources of the electronic device 101 (like the bus 110, the processor 120, or the memory 130) to at least one of the plurality of applications 147. The API 145 is an interface allowing the application 147 to control functions provided from the kernel 141 or the middleware 143. For example, the API 145 includes at least one interface or function (such as a command) for filing control, window control, image processing, or text control.

The I/O interface 150 serves as an interface that can, for example, transfer commands or data input from a user or other external devices to other component(s) of the electronic device 101. The I/O interface 150 can also output commands or data received from other component(s) of the electronic device 101 to the user or the other external device.

The display 160 includes, for example, a liquid crystal display (LCD), a light emitting diode (LED) display, an organic light emitting diode (OLED) display, a quantum-dot light emitting diode (QLED) display, a microelectromechanical systems (MEMS) display, or an electronic paper display. The display 160 can also be a depth-aware display, such as a multi-focal display. The display 160 is able to display, for example, various contents (such as text, images, videos, icons, or symbols) to the user. The display 160 can include a touchscreen and may receive, for example, a touch, gesture, proximity, or hovering input using an electronic pen or a body portion of the user.

The communication interface 170, for example, is able to set up communication between the electronic device 101 and an external electronic device (such as a first electronic device 102, a second electronic device 104, or a server 106). For example, the communication interface 170 can be connected with a network 162 or 164 through wireless or wired communication to communicate with the external electronic device. The communication interface 170 can be a wired or wireless transceiver or any other component for transmitting and receiving signals.

The wireless communication is able to use at least one of, for example, WiFi, long term evolution (LTE), long term evolution-advanced (LTE-A), 5th generation wireless system (5G), millimeter-wave or 60 GHz wireless communication, Wireless USB, code division multiple access (CDMA), wideband code division multiple access (WCDMA), universal mobile telecommunication system (UMTS), wireless broadband (WiBro), or global system for mobile communication (GSM), as a communication protocol. The wired connection can include, for example, at least one of a universal serial bus (USB), high definition multimedia interface (HDMI), recommended standard 232 (RS-232), or plain old telephone service (POTS). The network 162 or 164 includes at least one communication network, such as a computer network (like a local area network (LAN) or wide area network (WAN)), Internet, or a telephone network.

The electronic device 101 further includes one or more sensors 180 that can meter a physical quantity or detect an activation state of the electronic device 101 and convert metered or detected information into an electrical signal. For example, the sensor(s) 180 can include one or more cameras or other imaging sensors, which may be used to capture image frames of scenes. The sensor(s) 180 can also include one or more buttons for touch input, one or more microphones, a depth sensor, a gesture sensor, a gyroscope or gyro sensor, an air pressure sensor, a magnetic sensor or magnetometer, an acceleration sensor or accelerometer, a grip sensor, a proximity sensor, a color sensor (such as a red green blue (RGB) sensor), a bio-physical sensor, a temperature sensor, a humidity sensor, an illumination sensor, an ultraviolet (UV) sensor, an electromyography (EMG) sensor, an electroencephalogram (EEG) sensor, an electrocardiogram (ECG) sensor, an infrared (IR) sensor, an ultrasound sensor, an iris sensor, or a fingerprint sensor. Moreover, the sensor(s) 180 can include one or more position sensors, such as an inertial measurement unit (IMU) that can include one or more accelerometers, gyroscopes, and other components. In addition, the sensor(s) 180 can include a control circuit for controlling at least one of the sensors included here. Any of these sensor(s) 180 can be located within the electronic device 101.

In some embodiments, the electronic device 101 can represent a wearable device (such as a smart watch or a smart ring) or other device that can be used to locally or remotely monitor a subject. In other embodiments, the first external electronic device 102 and/or the second external electronic device 104 can represent at least one wearable device (such as a smart watch and/or a smart ring) and/or other device(s) that can be used to locally or remotely monitor a subject. In these other embodiments, the electronic device 101 could be directly connected with or communicate directly with the electronic device 102 and/or the electronic device 104 without involving a separate network, or connections or communications with the electronic device(s) 102, 104 may occur via one or more networks 162, 164.

The first and second external electronic devices 102 and 104 and the server 106 each can be a device of the same or a different type from the electronic device 101. According to certain embodiments of this disclosure, the server 106 includes a group of one or more servers. Also, according to certain embodiments of this disclosure, all or some of the operations executed on the electronic device 101 can be executed on another or multiple other electronic devices (such as the electronic devices 102 and 104 or server 106). Further, according to certain embodiments of this disclosure, when the electronic device 101 should perform some function or service automatically or at a request, the electronic device 101, instead of executing the function or service on its own or additionally, can request another device (such as electronic devices 102 and 104 or server 106) to perform at least some functions associated therewith. The other electronic device (such as electronic devices 102 and 104 or server 106) is able to execute the requested functions or additional functions and transfer a result of the execution to the electronic device 101. The electronic device 101 can provide a requested function or service by processing the received result as it is or additionally. To that end, a cloud computing, distributed computing, or client-server computing technique may be used, for example. While FIG. 1 shows that the electronic device 101 includes the communication interface 170 to communicate with the external electronic device 104 or server 106 via the network 162 or 164, the electronic device 101 may be independently operated without a separate communication function according to some embodiments of this disclosure.

The server 106 can include the same or similar components as the electronic device 101 (or a suitable subset thereof). The server 106 can support to drive the electronic device 101 by performing at least one of operations (or functions) implemented on the electronic device 101. For example, the server 106 can include a processing module or processor that may support the processor 120 implemented in the electronic device 101. As described below, the server 106 may perform one or more functions related to real-time sleep staging predictions using learned sleep structures, such as by training machine learning models and/or using trained machine learning models for real-time sleep staging predictions.

Although FIG. 1 illustrates one example of a network configuration 100 including an electronic device 101, various changes may be made to FIG. 1. For example, the network configuration 100 could include any number of each component in any suitable arrangement. In general, computing and communication systems come in a wide variety of configurations, and FIG. 1 does not limit the scope of this disclosure to any particular configuration. Also, while FIG. 1 illustrates one operational environment in which various features disclosed in this patent document can be used, these features could be used in any other suitable system.

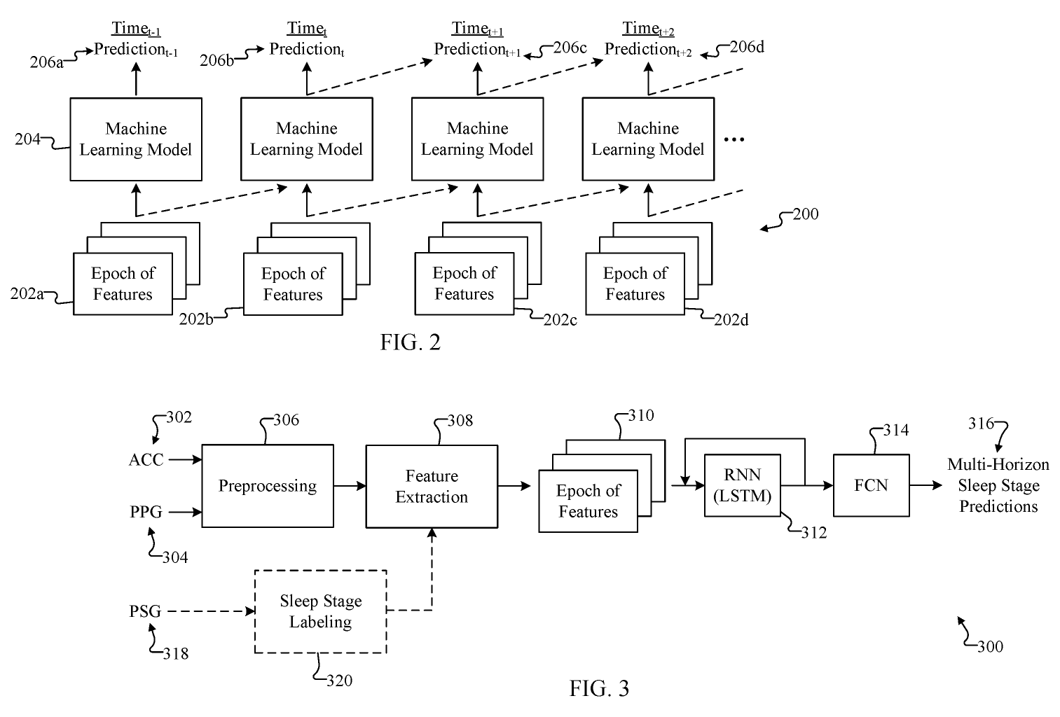

FIG. 2 illustrates an example process 200 for generating real-time sleep staging predictions using learned sleep structures in accordance with this disclosure. For ease of explanation, the process 200 shown in FIG. 2 is described as being performed using the electronic device 101 in the network configuration 100 shown in FIG. 1. However, the process 200 shown in FIG. 2 may be performed using any other suitable device(s) and in any other suitable system(s).

As shown in FIG. 2, the process 200 generally operates to obtain and process features of physiological information associated with a subject, where the features are divided into epochs 202a-202d. Each epoch 202a-202d can represent or be associated with a specified time interval, and a collection of epochs can be formed by a repeating time interval. For example, each epoch 202a-202d could represent a period of time equal to thirty seconds. For each epoch 202a-202d, physiological data can be collected or otherwise obtained, and features for that epoch 202a-202d can be derived from or otherwise generated using the physiological data of that epoch 202a-202d.

The physiological data can represent any suitable physiological-related information associated with a subject that could be indicative of or used to predict a current or future sleep stage of the subject. In some cases, the physiological data may include heart rate information of the subject, heart rate variability information of the subject, breathing rate information of the subject, movement information of the subject, or a combination thereof. In some embodiments, the physiological data could be preprocessed prior to identification of the features, such as by removing noise from the physiological data and/or normalizing the physiological data. The physiological data can also be obtained from any suitable source(s). As an example, at least some of the physiological data could be obtained from one or more wearable devices, such as a smart watch and/or a smart ring, worn by the subject. As another example, at least some of the physiological data could be obtained from one or more monitoring devices associated with the subject but not worn by the subject. For instance, one or more cameras could be used to remotely capture heart rate information (such as through RGB color changes) of the subject, and/or one or more smart home devices or other devices could use infrared illumination or other illumination to recognize sleeping movements of the subject at a distance.

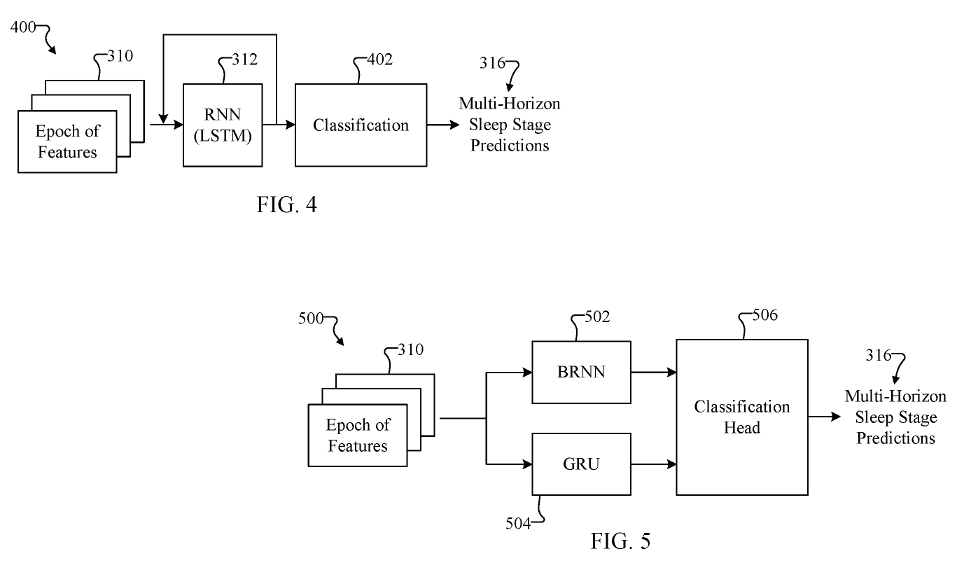

The features associated with the various epochs 202a-202d are provided to a trained machine learning model 204, which generally operates to process the features and generate predictions 206a-206d of current and future sleep stages of the subject. In some cases, the trained machine learning model 204 represents a time-series model that can process time-series data (as opposed to frequency data that lacks a time component) in order to generate the predictions 206a-206d. The trained machine learning model 204 can use any suitable machine learning architecture for processing features and generating sleep stage predictions, such as a recurrent neural network or other neural network. Example architectures for the trained machine learning model 204 are provided below and are described with reference to FIGS. 3 - 5.

In this example, it can be seen that the trained machine learning model 204 is used to repeatedly process the features associated with at least one current epoch and one or more prior epochs to generate real-time predictions of sleep stages of the subject during the at least one current epoch and one or more subsequent epochs. For example, during a first prediction time t–1, features in one or more epochs 202a can be provided to the trained machine learning model 204, and the trained machine learning model 204 can generate a prediction 206a based on the features in the one or more epochs 202a. This prediction 206a can represent a predicted sleep stage of the subject during the time interval(s) associated with the epoch(s) 202a.

During the next prediction time t, features in one or more epochs 202b can be provided to the trained machine learning model 204, and the trained machine learning model 204 can generate a prediction 206b based on the features in the one or more epochs 202b. This prediction 206b can represent a predicted sleep stage of the subject during the time interval(s) associated with the epoch(s) 202b. However, the trained machine learning model 204 can also generate the prediction 206b based on the features in the one or more prior epochs 202a. In other words, the prediction 206b for the current epoch(s) 202b can be based on both (i) the features of the physiological data associated with the current epoch(s) 202b and (ii) the features of the physiological data associated with the prior epoch(s) 202a. Moreover, during the prediction time t, the trained machine learning model 204 can generate at least one prediction 206c-206d of at least one future sleep stage of the subject. In other words, the trained machine learning model 204 can predict both the current sleep stage and one or more future sleep stages of the subject.

During the next prediction time t+1, features in one or more epochs 202c can be provided to the trained machine learning model 204, and the trained machine learning model 204 can generate a prediction 206c based on the features in the one or more epochs 202c. This prediction 206c can represent a predicted sleep stage of the subject during the time interval(s) associated with the epoch(s) 202c. Again, however, the trained machine learning model 204 can also generate the prediction 206c based on the features in the one or more prior epochs 202a and/or 202b. In other words, the prediction 206c for the current epoch(s) 202c can be based on both (i) the features of the physiological data associated with the current epoch(s) 202c and (ii) the features of the physiological data associated with the prior epoch(s) 202a, 202b. Moreover, during the prediction time t+1, the trained machine learning model 204 can generate at least one prediction 206d of at least one future sleep stage of the subject. In other words, the trained machine learning model 204 can predict both the current sleep stage and one or more future sleep stages of the subject.

During the next prediction time t+2, features in one or more epochs 202d can be provided to the trained machine learning model 204, and the trained machine learning model 204 can generate a prediction 206d based on the features in the one or more epochs 202d. This prediction 206d can represent a predicted sleep stage of the subject during the time interval(s) associated with the epoch(s) 202d. Again, however, the trained machine learning model 204 can also generate the prediction 206d based on the features in the one or more prior epochs 202a and/or 202b and/or 202c. In other words, the prediction 206d for the current epoch(s) 202d can be based on both (i) the features of the physiological data associated with the current epoch(s) 202d and (ii) the features of the physiological data associated with the prior epoch(s) 202a, 202b, 202c. Moreover, during the prediction time t+2, the trained machine learning model 204 can generate at least one prediction of at least one future sleep stage of the subject. In other words, the trained machine learning model 204 can predict both the current sleep stage and one or more future sleep stages of the subject.

By repeatedly using the trained machine learning model 204 in this manner, the trained machine learning model 204 can be used to generate real-time predictions of the current sleep stage of the subject, as well as predicted future sleep stages of the subject. As can be seen here, the trained machine learning model 204 is not merely used to process the features for each individual epoch 202a-202d and make a sleep stage prediction for that individual epoch. Rather, the trained machine learning model 204 can be used to process features for both current and prior epochs, as well as generate predictions for both current and future epochs. In some embodiments, the features for both current and prior epochs can be input to the trained machine learning model 204 as a “long context” input, meaning the features for these epochs can provide context to the trained machine learning model 204 during the generation of the sleep stage predictions. The number of epochs for which features are provided to the trained machine learning model 204 as input can be referred to as the context size of the trained machine learning model 204. Also, in some embodiments, the trained machine learning model 204 can be said to support or perform a multi-horizon prediction task in which each horizon is associated with a different prediction, namely one horizon for the prediction of the subject’s current sleep stage and one or more additional horizons for the prediction(s) of the subject’s future sleep stage(s). As a result, the trained machine learning model 204 can be said to implement a multi-horizon prediction framework (M-HPF). Here, the trained machine learning model 204 can generate the predictions for all of these sleep stages at once. The number of future sleep stage predictions that are generated by the trained machine learning model 204 can be referred to as the prediction length of the trained machine learning model 204.

As described in more detail below, in some embodiments, the trained machine learning model 204 can be trained using polysomnography data, either as ground truth data or to derive ground truth data. Polysomnography data is often collected during sleep studies, allowing the polysomnography data to be used to accurately identify the stages of sleep of the people involved in the sleep studies. Physiological data (such as heart rate, heart rate variability, breathing rate, and/or movement information) can be used as training data, and this physiological data can be extracted from or otherwise identified using the polysomnography data. Labels of actual sleep stages can also be identified by or derived from the polysomnography data. The training data can be provided to a machine learning model being trained, and outputs (predictions of sleep stages) generated by the machine learning model can be compared to the ground truths. Differences between the predictions and the ground truths can be used to calculate a loss of the machine learning model, and parameters (such as weights) of the machine learning model can be adjusted based on the loss. The adjusted machine learning model can be used to process the same training data or different training data, and the loss can be updated. This can continue for any number of iterations, typically until the loss reaches a suitably-low value (such as a value below a threshold) or some other stopping criterion or criteria are met (such as elapsed training time or number of training iterations).

Since the trained machine learning model 204 here can be trained to predict both the current sleep stage and at least one future sleep stage of a subject, the trained machine learning model 204 can learn during the training to recognize transitions between different sleep stages of people. As a result, the trained machine learning model 204 can be used during inferencing to process features of physiological data for a subject across a number of epochs in order to predict both current and future sleep stages of the subject. Moreover, the trained machine learning model 204 can learn during the training to recognize the overall structure of sleep by people. That is, people generally experience different stages of sleep, such as light sleep, deep sleep, and rapid eye movement (REM) sleep, during the night. People typically will not randomly jump between any two stages. Rather, people typically transition between certain sleep stages, and various probabilities for transitioning between those certain sleep stages can be known or derived. The trained machine learning model 204 can learn the overall structure of sleep by the people associated with the training data, such as by learning the various probabilities for transitioning between various sleep stages. This effectively allows the trained machine learning model 204 to learn the high-level temporal structures of sleep (typically on the timescale of hours, but other more-granular temporal structures of sleep could also be learned). Overall, this approach allows the trained machine learning model 204 to generate sleep stage predictions more accurately.

The sleep stage predictions generated using the trained machine learning model 204 may be used in any suitable manner. For example, the sleep stage predictions could be used by an electronic device 101, 102, 104 or other device to predict a sleep score for the subject (where the sleep score represents an overall score of the quality of the subject’s sleep) and/or to generate a graphical display that illustrates the sleep stages of the subject during the night. As another example, the sleep stage predictions could be used by an electronic device 101, 102, 104 or other device to derive one or more health predictions for the subject, such as to estimate the overall quality of the subject’s sleep or generate one or more predictions of whether the subject might be suffering from one or more issues. These types of predictions could be used to provide advice to the subject, such as by analyzing the subject’s sleep-related information and identifying one or more potential sleep interventions that could be applied in order to improve the subject’s sleep quality and/or morning cognitive abilities.

As yet another example, the sleep stage predictions could be used by an electronic device 101, 102, 104 or other device to control one or more operations of that device or another device. As a particular example, the electronic device 101, 102, 104 or other device could implement a smart alarm feature in which an alarm sound is generated at a set or variable time only when a subject is determined to be in a light stage of sleep (in order to be less disruptive). The smart alarm feature could refrain from generating an alarm sound when the subject is determined to be in a deep or REM stage of sleep. As another particular example, the electronic device 101, 102, 104 or other device could implement a deep sleep enhancement function in which the device, after detecting that the subject is in deep sleep, can enable one or more interventions (such as slow-wave entrainment) using sounds and/or vibrations. In some cases, the sounds and/or vibrations could be provided by a smart watch or other wearable device, which may or may not be the same device that predicts the sleep stages of the subject.

Although FIG. 2 illustrates one example of a process 200 for generating real-time sleep staging predictions using learned sleep structures, various changes may be made to FIG. 2. For example, epochs could be processed individually by the trained machine learning model 204, rather than in groups. Also, the trained machine learning model 204 need not use one or more prior epochs and/or need not generate one or more future predictions for every epoch or collection of epochs.

FIGS. 3 - 5 illustrate example architectures 300, 400, 500 for generating real-time sleep staging predictions using learned sleep structures in accordance with this disclosure. More specifically, FIGS. 3 - 5 illustrate example architectures 300, 400, 500 that could be used to implement the machine learning model 204 of FIG. 2. For ease of explanation, the architectures 300, 400, 500 shown in FIGS. 3 - 5 are described as being used in the process 200 of FIG. 2, which could be implemented using the electronic device 101 in the network configuration 100 shown in FIG. 1. However, the architectures 300, 400, 500 shown in FIGS. 3 - 5 may be implemented using any other suitable device(s) and in any other suitable system(s) and may be used in any other suitable process(es).

As shown in FIG. 3, the architecture 300 receives and processes physiological data associated with a subject. The physiological data in this example includes acceleration (ACC) data 302 and photoplethysmography (PPG) data 304. The acceleration data 302 can represent acceleration or other movement data associated with the subject, such as data collected using at least one IMU, gyroscope, or accelerometer. The acceleration data 302 could help to identify movement or breathing rate of the subject during sleep. The photoplethysmography data 304 can represent blood flow or other heart-related data associated with the subject, such as data collected using at least one pulse monitor or pulse oximeter. The photoplethysmography data 304 could help to identify heart rate or heart rate variability of the subject during sleep. Note that these forms of the physiological data are examples only and that other or additional types of physiological data may be obtained here.

The physiological data is provided to a preprocessing function 306, which generally operates to preprocess the physiological data prior to additional functions. The preprocessing function 306 could perform any desired preprocessing of the physiological data. For example, the preprocessing function 306 could denoise the physiological data, such as by removing outlier values or other incorrect or anomalous physiological data values. The preprocessing function 306 could also or alternatively normalize the physiological data, such as by scaling heart rate, heart rate variability, breathing rate, movement, and/or other physiological information associated with the subject so that each of these characteristics is expressed as a value within an expected range of values.

The preprocessed physiological data is provided to a feature extraction function 308, which generally operates to transform raw physiological data into compact and meaningful numerical representations (feature vectors) that capture essential characteristics of the physiological data. For example, the feature extraction function 308 can be trained to convert the preprocessed physiological data from the preprocessing function 306 into feature vectors, which represent the important or useful characteristics of the physiological data in a latent feature space. The feature extraction function 308 can use any suitable technique(s) to extract features from physiological data. The preprocessed physiological data here can be associated with different epochs. Thus, the features produced by the feature extraction function 308 can form multiple epochs 310 of features. In some cases, each epoch 310 of features may be generated using the physiological data obtained during a specified interval of time, such as within a thirty second interval of time. This interval can repeat throughout a subject’s sleep, thereby leading to the generation of a long series of epochs 310 of features, such as is shown in FIG. 2.

The epochs 310 of features are provided to a recurrent neural network (RNN) 312, which process the epochs 310 of features in order to generate outputs that are provided to a fully-connected network (FCN) 314. The recurrent neural network 312 represents a neural network that is designed to process sequential data, making it suitable for use in processing sequential epochs 310 of features associated with a subject’s sleep. The recurrent neural network 312 typically includes neurons in which the outputs of the neurons are fed back as inputs to the neurons so that the recurrent neural network 312 can learn and use temporal dependencies. The recurrent neural network 312 can be implemented in any suitable manner, such as by using at least one long short-term memory (LSTM). The fully-connected network 314 uses the outputs of the recurrent neural network 312 to generate sleep stage predictions 316. The fully-connected network 314 represents a neural network having multiple layers or neurons, where each neuron in one layer is connected to (i) all neurons in the preceding layer (if any) and (ii) all neurons in the succeeding layer (if any). The fully-connected network 314 can be implemented in any suitable manner, such as by using any suitable number of neurons in any suitable number of layers.

As described above, both current and prior epochs 310 of features can be provided as input to the recurrent neural network 312 and processed using the recurrent neural network 312. In some cases, the number of epochs 310 used as input to the recurrent neural network 312 represents the context size of the recurrent neural network 312 and can be denoted N, and the epochs 310 of features used as input to the recurrent neural network 312 can form a long-context input. Also, as described above, the architecture 300 can be used to generate multi-horizon predictions 316, meaning predictions of both the current sleep stage of the subject (for one or more current epochs 310) and one or more future sleep stages of the subject (for one or more future epochs 310). The number of predictions 316 made by the architecture 300 can be denoted M, which can denote the number of horizons in which predictions 316 are included in the output of the fully-connected network 314.

To train this architecture 300, polysomnography (PSG) data 318 can be obtained, such as polysomnography data obtained during various sleep studies of a number of people. The polysomnography data 318 could be provided to a sleep stage labeling function 320, which can process the polysomnography data 318 and identify actual sleep stages of the people associated with the polysomnography data 318 over time. Various ways of performing this type of polysomnography data processing are known and can be used here. By separating the polysomnography data 318 into repeating intervals over time and processing at least some of the polysomnography data 318 in those repeating intervals with the feature extraction function 308, the resulting epochs 310 can represent training data, and the epochs 310 can be labeled using the correct sleep stage predictions as generated by the sleep stage labeling function 320.

Thus, during training, the epochs 310 containing training data can be processed by the recurrent neural network 312, and the fully-connected network 314 can generate predictions 316 that can be compared to the labels (ground truths) generated by the sleep stage labeling function 320. Differences between the predictions 316 and the labels can be used to estimate a loss of the architecture 300, and the architecture 300 can be adjusted (such as by modifying weights or other parameters of the recurrent neural network 312 and/or the fully-connected network 314). This process can be repeated until the loss is below a threshold or one or more other stopping criteria are satisfied.

The use of the recurrent neural network 312 and the ability to generate predictions of future sleep stages facilitate the learning of high-level structures of various sleep stages. The recurrent neural network 312 enables the architecture 300 to learn high-level patterns in time-series signals by introducing the ability to account for information from the past. This helps to account for the fact that a given sleep stage is not necessarily independent of the preceding sleep stage(s).

In the example shown in FIG. 3, the fully-connected network 314 is used as a classifier to identify the predicted sleep stages of a subject based on the output of the recurrent neural network 312. However, other approaches could be used. For instance, as shown in FIG. 4, the output of the recurrent neural network 312 could be provided to a classification function 402. The output of the recurrent neural network 312 could include sleep stage probabilities indicating a probability of each sleep stage being associated with a given epoch or collection of epochs. The classification function 402 could process these probabilities in order to generate the final sleep stage predictions 316, such as by identifying which sleep stage predictions have the largest probabilities.

Also, in the examples shown in FIGS. 3 and 4, at least one long short-term memory is described as being used as the recurrent neural network 312. However, other implementations are possible. For example, in FIG. 5, the recurrent neural network 312 could be implemented using at least one bidirectional RNN (BRNN) 502 and/or at least one gated recurrent unit (GRU) 504. Designs for both of these structures are known and could be trained (such as in the manner recited above) to implement the desired functionality. In FIG. 5, a specific implementation of the classification function 402 is also shown, namely a classification head 506. The classification head 506 could represent the final layer or layers of a neural network (such as one implementing the recurrent neural network 312) and can be used to classify probabilities generated by earlier layers of the neural network into the final sleep stage predictions 316.

Although FIGS. 3 - 5 illustrate examples of architectures 300, 400, 500 for generating real-time sleep staging predictions 316 using learned sleep structures, various changes may be made to FIGS. 3 - 5. For example, other implementations of the machine learning model 204 could be used, including those that use components other than a recurrent network to learn how to recognize transitions between different sleep stages and how to recognize an overall structure of sleep.

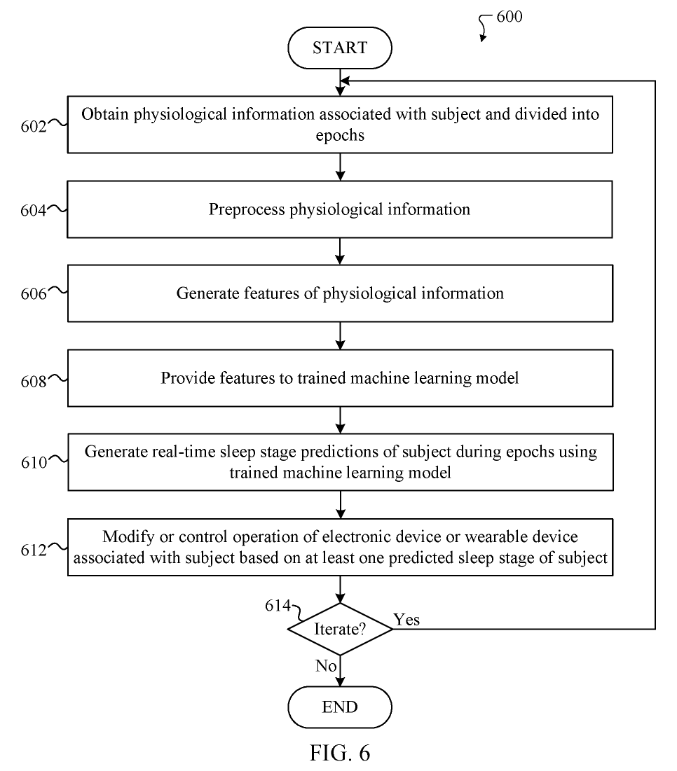

FIG. 6 illustrates an example method 600 for generating real-time sleep staging predictions using learned sleep structures in accordance with this disclosure. For ease of explanation, the method 600 shown in FIG. 6 is described as being performed using the electronic device 101 in the network configuration 100 shown in FIG. 1. However, the method 600 shown in FIG. 6 may be performed using any other suitable device(s) (such as the server 106) and in any other suitable system(s).

As shown in FIG. 6, physiological information associated with a subject and divided into epochs is obtained at step 602. This may include, for example, the processor 120 of the electronic device 101 obtaining physiological information from one or more wearable devices (such as a smart watch and/or a smart ring) worn by the subject. This may also or alternatively include the processor 120 of the electronic device 101 obtaining physiological information from one or more remote sensors monitoring the subject. Depending on the implementation, the device that obtains and uses the physiological information may or may not represent the device(s) used to originally capture the physiological information. In general, the physiological information can be obtained from any suitable source(s). In some cases, the physiological information can include heart rate information of the subject, heart rate variability information of the subject, breathing rate information of the subject, movement information of the subject, or a combination thereof. Also, in some cases, the epochs can be defined by a repeating time interval, such as a thirty-second window. The physiological information may be obtained already divided into epochs, or the physiological information may be divided into epochs after being obtained.

The physiological information may optionally be preprocessed at step 604. This may include, for example, the processor 120 of the electronic device 101 removing noise from the physiological information and/or normalizing the physiological information using the preprocessing function 306. In general, any desired preprocessing of the physiological information may be performed here.

Features of the physiological information are generated at step 606. This may include, for example, the processor 120 of the electronic device 101 processing the physiological information (or the preprocessed version thereof) using the feature extraction function 308 in order to generate feature vectors associated with the physiological information. The features can be generated for the physiological information obtained during each epoch to create epochs 202a-202d, 310 of features. The features could be generated for different collections of time intervals using the physiological information collected during the different collections of time intervals, such as by processing the features in groups of epochs 202a-202d, 310.

The features are provided to a trained machine learning model at step 608. This may include, for example, the processor 120 of the electronic device 101 providing the epochs 202a-202d, 310 of features to the trained machine learning model 204. The trained machine learning model 204 can be trained to recognize transitions between different sleep stages of people and an overall structure of sleep by the people. In some cases, polysomnography data could have been used as ground truth data or to generate the ground truth data used during training of the trained machine learning model 204, which allows the trained machine learning model 204 to learn how to label the epochs 202a-202d, 310 with real-time sleep stage predictions.

Real-time sleep stage predictions of the subject during the epochs are generated using the trained machine learning model at step 610. This may include, for example, the processor 120 of the electronic device 101 using the trained machine learning model 204 to process the epochs 202a-202d, 310 and generate predictions 206a-206d, 316 of both current and future sleep stages of the subject. In some cases, the trained machine learning model 204 can generate real-time sleep stage predictions 206a-206d, 316 for at least one current epoch using the features associated with the at least one current epoch and the features associated with one or more prior epochs. The real-time sleep stage predictions 206a-206d, 316 for the at least one current epoch could include (i) a predicted sleep stage of the subject during the at least one current epoch and (ii) at least one predicted future sleep stage of the subject during one or more subsequent epochs following the at least one current epoch. Here, for at least some of the time intervals associated with the epochs, the trained machine learning model 204 can process the features associated with the at least one current epoch and one or more prior epochs and generate real-time predictions of sleep stages of the subject during the at least one current epoch and one or more subsequent epochs.

The real-time sleep stage predictions of the subject may be used in any suitable manner. In this example, operation of an electronic device or at least one wearable device associated with the subject can be modified or controlled based on at least one predicted sleep stage of the subject at step 612. This may include, for example, the processor 120 of the electronic device 101 using the real-time sleep stage predictions to predict and initiate display or otherwise use of a sleep score for the subject, derive one or more health predictions for the subject, provide advice to the subject, implement a smart alarm feature, or implement a deep sleep enhancement function as described above.

A determination is made whether to iterate the process at step 614. This may include, for example, the processor 120 of the electronic device 101 determining whether additional physiological information remains to be processed for the subject. If so, the process can return to an earlier step (such as step 602 in this example) to process the additional physiological information.

Although FIG. 6 illustrates one example of a method 600 for generating real-time sleep staging predictions using learned sleep structures, various changes may be made to FIG. 6. For example, while shown as a series of steps, various steps in FIG. 6 could overlap, occur in parallel, occur in a different order, or occur any number of times (including zero times).

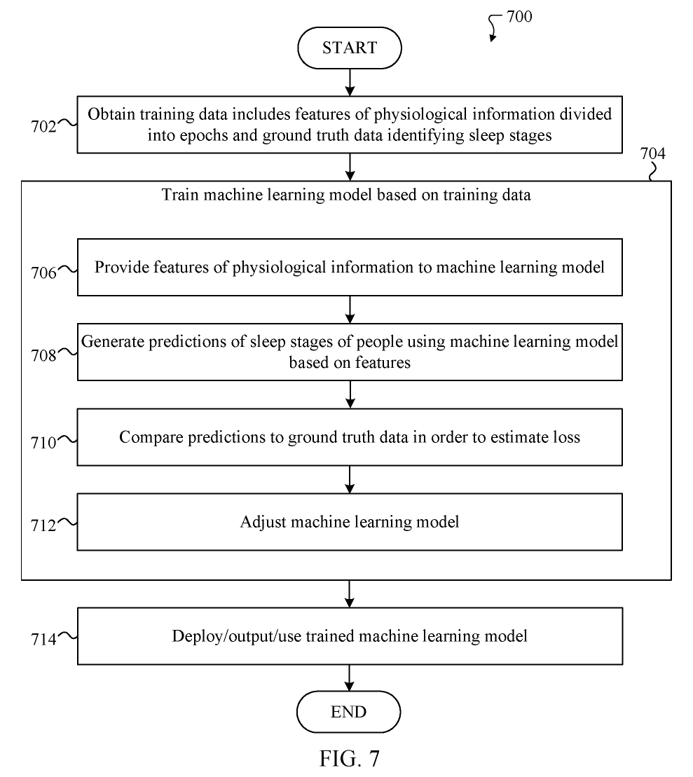

FIG. 7 illustrates an example method 700 for training a machine learning model to generate real-time sleep staging predictions using learned sleep structures in accordance with this disclosure. For ease of explanation, the method 700 shown in FIG. 7 is described as being performed using the server 106 in the network configuration 100 shown in FIG. 1. However, the method 700 shown in FIG. 7 may be performed using any other suitable device(s) (such as the electronic device 101) and in any other suitable system(s).

As shown in FIG. 7, training data is obtained at step 702. This may include, for example, the processor 120 of the server 106 obtaining training data that includes features of physiological information associated with a number of people and ground truth data identifying sleep stages of the people. The physiological information can be divided into epochs associated with different periods of time. Note that the training data and the ground truth data could be generated by the server 106 (such as based on polysomnography data 318) or obtained from any suitable source(s). In some cases, the physiological information includes heart rate information of the people, heart rate variability information of the people, breathing rate information of the people, movement information of the people, or a combination thereof. Also, in some cases, the polysomnography data can be used as the ground truth data or to generate the ground truth data during the training of the machine learning model 204.

A machine learning model is trained based on the training data at step 704. This may include, for example, the processor 120 of the server 106 training the machine learning model 204 to repeatedly predict, based on features of physiological information associated with a specified subject during at least one current epoch and one or more prior epochs, (i) a current sleep stage of the specified subject during the at least one current epoch and (ii) one or more predicted future sleep stages of the specified subject during one or more subsequent epochs following the at least one current epoch. As part of this process, the machine learning model 204 can be trained to recognize transitions between different sleep stages of the people and an overall structure of sleep by the people. This can allow the machine learning model 204 to learn how, for at least some of the epochs associated with a specified subject, to process the features associated with at least one current epoch and one or more prior epochs and generate real-time predictions of sleep stages of the specified subject during the at least one current epoch and one or more subsequent epochs.

In some cases, this training may occur as follows. The features of the physiological information associated with the people can be provided to the machine learning model at step 706, and predictions of sleep stages of the people can be generated using the machine learning model based on the features of the physiological information associated with the people at step 708. This may include, for example, the processor 120 of the server 106 providing the epochs 202a-202d, 310 of features (the training data) to the machine learning model 204 and generating predictions 206a-206d, 316 of the sleep stages of the people. The predictions can be compared to the ground truth data in order to estimate a loss at step 710, and the machine learning model can be adjusted based on the loss at step 712. This may include, for example, the processor 120 of the server 106 using a loss function to calculate the loss of the machine learning model 204 based on differences between the generated predictions 206a-206d, 316 and the ground truth predictions. This may also include the processor 120 of the server 106 using backpropagation or other technique(s) to modify weights or other parameters of the machine learning model 204. Note that this is typically done iteratively, meaning steps 706-712 can be performed repeatedly until at least one stopping criterion is satisfied, such as the calculated loss being below a threshold value.

Once adequately trained, the machine learning model can be deployed, output, or used in some manner at step 714. This may include, for example, the processor 120 of the server 106 using the trained machine learning model 204 as part of the process 200 or providing the trained machine learning model 204 to one or more other devices (such as the electronic device 101) for use during the process 200.

Although FIG. 7 illustrates one example of a method 700 for training a machine learning model to generate real-time sleep staging predictions using learned sleep structures, various changes may be made to FIG. 7. For example, while shown as a series of steps, various steps in FIG. 7 could overlap, occur in parallel, occur in a different order, or occur any number of times (including zero times).

It should be noted that the functions shown in or described with respect to the figures can be implemented in an electronic device 101, 102, 104, server 106, or other device(s) in any suitable manner. For example, in some embodiments, at least some of the functions shown in or described with respect to the figures can be implemented or supported using one or more software applications or other software instructions that are executed by the processor 120 of the electronic device 101, 102, 104, server 106, or other device(s). In other embodiments, at least some of the functions shown in or described with respect to the figures can be implemented or supported using dedicated hardware components. In general, the functions shown in or described with respect to the figures can be performed using any suitable hardware or any suitable combination of hardware and software/firmware instructions. Also, the functions shown in or described with respect to the figures can be performed by a single device or by multiple devices.

Although this disclosure has been described with example embodiments, various changes and modifications may be suggested to one skilled in the art. It is intended that this disclosure encompass such changes and modifications as fall within the scope of the appended claims.

What is claimed is:

1. A method comprising:
obtaining, using at least one processing device, physiological information associated with a subject, wherein the physiological information is divided into epochs associated with different periods of time;
generating, using the at least one processing device, features of the physiological information;
providing, using the at least one processing device, the features to a trained machine learning model; and
generating, using the at least one processing device, real-time sleep stage predictions of the subject during the epochs using the trained machine learning model;
wherein the trained machine learning model generates the real-time sleep stage predictions for at least one current epoch using the features associated with the at least one current epoch and the features associated with one or more prior epochs, the real-time sleep stage predictions for the at least one current epoch comprising (i) a predicted sleep stage of the subject during the at least one current epoch and (ii) at least one predicted future sleep stage of the subject during one or more subsequent epochs following the at least one current epoch.

2. The method of claim 1, wherein:
the epochs are defined by a repeating time interval;
the features are generated for different collections of time intervals using the physiological information collected during the different collections of time intervals; and
for at least some of the time intervals, the trained machine learning model processes the features associated with the at least one current epoch and the one or more prior epochs and generates real-time predictions of sleep stages of the subject during the at least one current epoch and the one or more subsequent epochs.

3. The method of claim 1, wherein the trained machine learning model is trained to recognize transitions between different sleep stages of people and an overall structure of sleep by the people.

4. The method of claim 1, wherein polysomnography data is used as ground truth data or to generate the ground truth data during training of the trained machine learning model so that the trained machine learning model learns to label the epochs with the real-time sleep stage predictions.

5. The method of claim 1, wherein the physiological information comprises heart rate information of the subject, heart rate variability information of the subject, breathing rate information of the subject, movement information of the subject, or a combination thereof.

6. The method of claim 1, further comprising:
preprocessing the physiological information to generate preprocessed physiological information;
wherein generating the features comprises generating the features using the preprocessed physiological information; and
wherein preprocessing the physiological information comprises at least one of: removing noise from the physiological information or normalizing the physiological information.

7. The method of claim 1, further comprising:
modifying or controlling operation of an electronic device or at least one wearable device associated with the subject based on at least one of the predicted sleep stage of the subject during the current epoch or the at least one predicted future sleep stage of the subject during the one or more subsequent epochs.

8. An apparatus comprising:
at least one processing device configured to:
obtain physiological information associated with a subject, wherein the physiological information is divided into epochs associated with different periods of time;
generate features of the physiological information;
provide the features to a trained machine learning model; and
generate real-time sleep stage predictions of the subject during the epochs using the trained machine learning model;
wherein the trained machine learning model is configured to generate the real-time sleep stage predictions for at least one current epoch using the features associated with the at least one current epoch and the features associated with the one or more prior epochs, the real-time sleep stage predictions for the at least one current epoch comprising (i) a predicted sleep stage of the subject during the at least one current epoch and (ii) at least one predicted future sleep stage of the subject during one or more subsequent epochs following the at least one current epoch.

9. The apparatus of claim 8, wherein:
the epochs are defined by a repeating time interval;
the at least one processing device is configured to generate the features for different collections of time intervals using the physiological information collected during the different collections of time intervals; and
for at least some of the time intervals, the trained machine learning model is configured to process the features associated with the at least one current epoch and one or more prior epochs and generate real-time predictions of sleep stages of the subject during the at least one current epoch and the one or more subsequent epochs.

10. The apparatus of claim 8, wherein the trained machine learning model is trained to recognize transitions between different sleep stages of people and an overall structure of sleep by the people.

11. The apparatus of claim 8, wherein polysomnography data is used as ground truth data or to generate the ground truth data during training of the trained machine learning model so that the trained machine learning model learns to label the epochs with the real-time sleep stage predictions.

12. The apparatus of claim 8, wherein the physiological information comprises heart rate information of the subject, heart rate variability information of the subject, breathing rate information of the subject, movement information of the subject, or a combination thereof.

13. The apparatus of claim 8, wherein:
the at least one processing device is further configured to preprocess the physiological information to generate preprocessed physiological information;
the at least one processing device is configured to generate the features using the preprocessed physiological information; and
to preprocess the physiological information, the at least one processing device is configured to at least one of: remove noise from the physiological information or normalize the physiological information.

14. The apparatus of claim 8, wherein the at least one processing device is further configured to modify or control operation of an electronic device or at least one wearable device associated with the subject based on at least one of the predicted sleep stage of the subject during the current epoch or the at least one predicted future sleep stage of the subject during the one or more subsequent epochs.

15. A method comprising:
obtaining, using at least one processing device of an electronic device, training data comprising (i) features of physiological information associated with people, the physiological information divided into epochs associated with different periods of time, and (ii) ground truth data identifying sleep stages of the people; and
training, using the at least one processing device, a machine learning model based on the training data;
wherein the machine learning model is trained to repeatedly predict, based on features of physiological information associated with a specified subject during at least one current epoch and one or more prior epochs, (i) a current sleep stage of the specified subject during the at least one current epoch and (ii) one or more predicted future sleep stages of the specified subject during one or more subsequent epochs following the at least one current epoch.

16. The method of claim 15, wherein the machine learning model is trained to recognize transitions between different sleep stages of the people and an overall structure of sleep by the people.

17. The method of claim 15, wherein training the machine learning model comprises:
providing the features of the physiological information associated with the people to the machine learning model;
generating predictions of sleep stages of the people using the machine learning model based on the features of the physiological information associated with the people;
comparing the predictions to the ground truth data in order to estimate a loss; and
adjusting the machine learning model until the loss is below a threshold.

18. The method of claim 15, wherein the machine learning model is trained, for at least some of the epochs associated with the specified subject, to process the features associated with the at least one current epoch and the one or more prior epochs and generate real-time predictions of sleep stages of the specified subject during the at least one current epoch and the one or more subsequent epochs.

19. The method of claim 15, wherein polysomnography data is used as the ground truth data or to generate the ground truth data during the training of the machine learning model.

20. The method of claim 15, wherein the physiological information comprises heart rate information of the people, heart rate variability information of the people, breathing rate information of the people, movement information of the people, or a combination thereof.
